(19) United States
(12) Patent Application Publication
(10) Pub. No.: US 2006/0200061 A1
(43) Pub. Date: Sep. 7, 2006

(54) Stabilization and traction apparatus and method for non-operative treatment of distal radius and upper extremity fractures

(76) Inventor: Blaine L. Warkentine, Long Beach, CA (US)

Correspondence Address: Steven M. Haas;Fay, Sharpe, Fagan, Minnich & McKee, LLP, Seventh Floor, 1100 Superior Avenue, Cleveland, OH, 44114-2579, US

(21) Appl. No.: 11/366,258

(22) Filed: Mar. 2, 2006

Related U.S. Application Data
(60) Provisional application No. 60/658,413, filed on Mar. 3, 2005.

(51) Int. Cl. A61F 5/00

(52) U.S. Cl. 602/32; 602/36

(57) ABSTRACT

A stabilization and traction device for non-operative treatment of distal radius and upper extremity fractures includes a base, an arm anchorage device connected to the base and a traction device connected to the base at a location spaced-apart from the arm anchorage device. The traction device includes a traction strap that engages a patient's wrist and/or fingers and that can be selectively tensioned. The device is used for reduction of distal radius or other fractures by securing a patient's upper arm and applying sufficient longitudinal traction to reduce the fracture. The device can be configured to apply palmer translation in combination with the longitudinal traction. The device is also configured to facilitate splinting/casting operations while the patient's arm is immobilized in the device. Use of the device is described for non-operative treatment of distal radius and upper extremity fractures.

### CROSS-REFERENCE TO RELATED APPLICATION

This application claims priority from and benefit of the filing date of U.S. provisional application Ser. No. 60/658,413 filed Mar. 3, 2005, and the disclosure of provisional application Ser. No. 60/658,413 is hereby expressly incorporated by reference into the present application.

### BACKGROUND

Reduction of distal radius fractures and other non-operative treatment of upper extremity fractures have heretofore been accomplished using a variety of different devices/methods, none of which have been deemed entirely satisfactory. In some cases devices such as intravenous support poles, weights, and the like have been assembled into makeshift reduction devices. In many such known devices, the patient's forearm is held vertically, without any support against lateral movement and without any visible indicia means for allowing the healthcare professional to assess the amount of traction applied to the forearm. Known devices have also not been found to provide suitable means for inducing controlled palmer translation or ulnar deviation in combination with the traction. Finally, some known devices do not facilitate application of a splint once the fracture is reduced.

### SUMMARY

In accordance with a first aspect of the present development, a stabilization and traction apparatus includes a base; an arm anchorage device connected to the base, the arm anchorage device adapted to engage an associated patient upper arm; and a traction device connected to the base. The traction device comprises a traction strap connected thereto and a tensioner for selectively tensioning the strap.

In accordance with another aspect of the present development, a method for non-operative treatment of an upper extremity fracture comprises: supporting on a base at least one of: (i) a patient hand; (ii) a patient wrist; (iii) at least part of a patient forearm; engaging a patient upper with arm anchorage device that is connected to the base; connecting a traction strap of a traction device to at least one of: (i) the patient hand; (ii) the patient wrist; and, operating the traction device to tension the traction strap, wherein the arm anchorage device restrains the patient upper arm against movement in response to the tension in the traction strap.

### BRIEF DESCRIPTION OF DRAWINGS

The development comprises various components and arrangements of components and/or various steps and arrangements of steps, preferred embodiments of which are disclosed in connection with the drawings, wherein:

FIG. 1 is an isometric view of a stabilization and traction apparatus formed in accordance with the present development and showing a patient's arm operatively engaged therewith for a fracture reduction and splinting/casting procedure in accordance with the present development;

FIG. 2 is similar to FIG. 1, without showing the patient's arm;

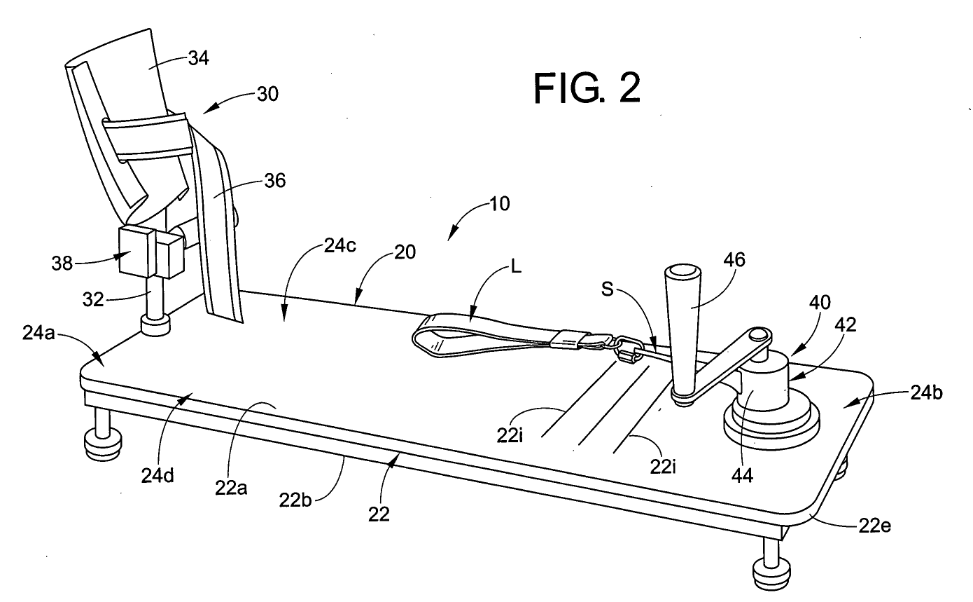

FIG. 3 is a side view of the device of FIG. 2;

FIG. 3A is identical to FIG. 3 except that it shows an alternative traction strap including finger cuffs;

FIG. 4 is a rear view of the device of FIG. 2;

FIG. 5 is a top view of the device of FIG. 2;

FIG. 6 is similar to FIG. 5, but shows a patient's arm operatively engaged with the device for a fracture reduction and splinting procedure in accordance with the present development.

### DETAILED DESCRIPTION

With reference to the drawings, a stabilization and traction apparatus/device 10 formed in accordance with the present development comprises a base 20, an arm anchorage device 30 connected to the base and a traction device 40 connected to the base at a location spaced-apart from the arm anchorage device 30.

In the most general terms, the device 10 is useful for stabilizing and applying traction for non-operative treatment of distal radius and upper extremity fractures by securing a patient's upper arm UA in a fixed location and applying sufficient longitudinal traction to the patient forearm FA, e.g., to reduce a distal radius or other fracture. The device 10 can also be configured to apply palmer translation in combination with the longitudinal traction, and facilitates application of ulnar deviation by stabilizing the upper arm UA and forearm FA. The device 10 is also configured to facilitate splinting/casting operations while the patient's arm A is immobilized in the device 10.

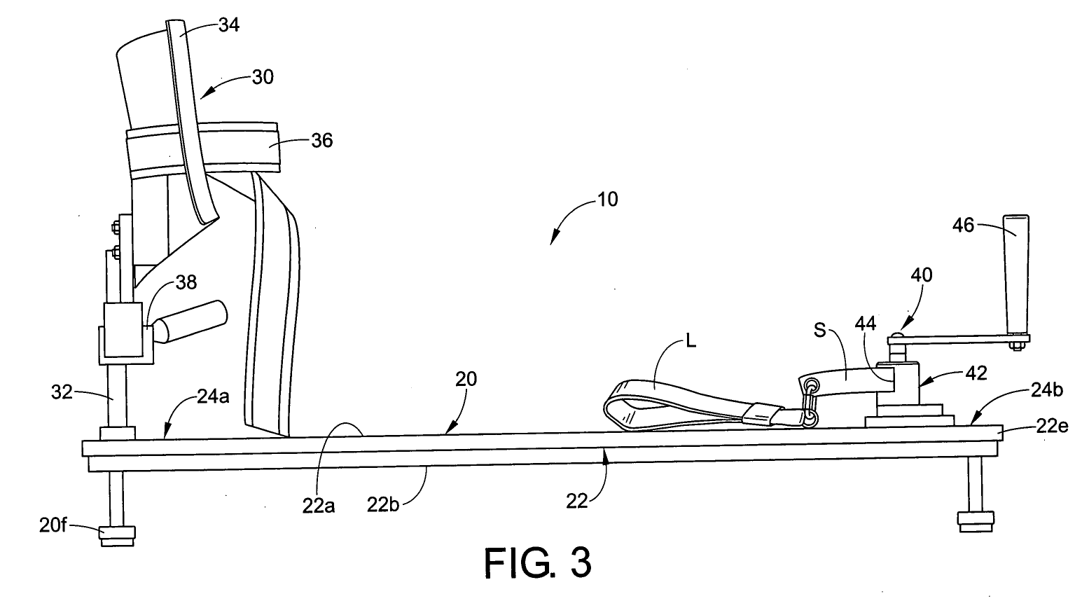

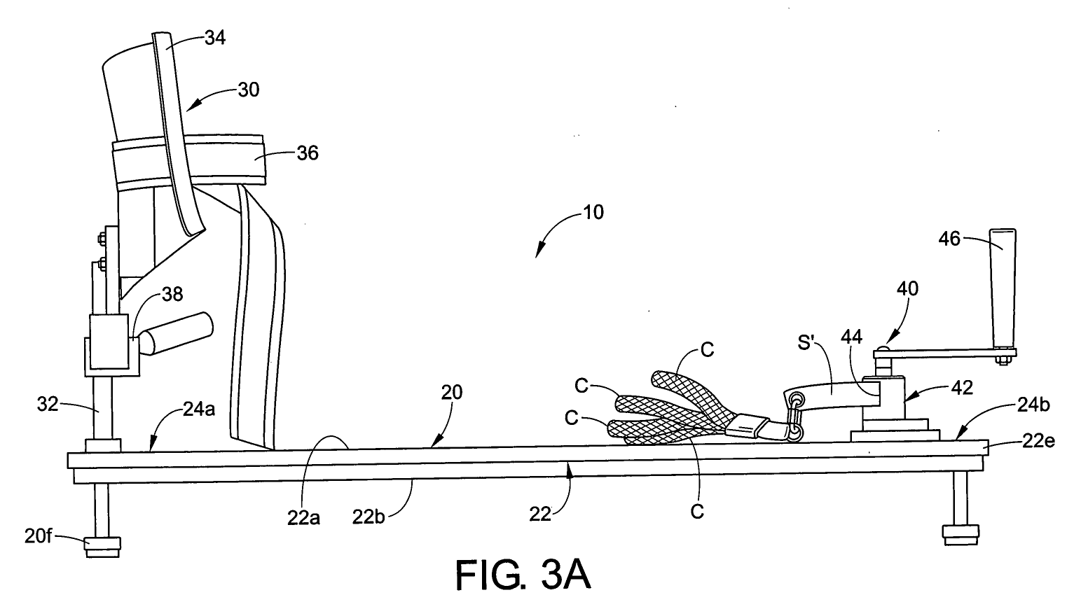

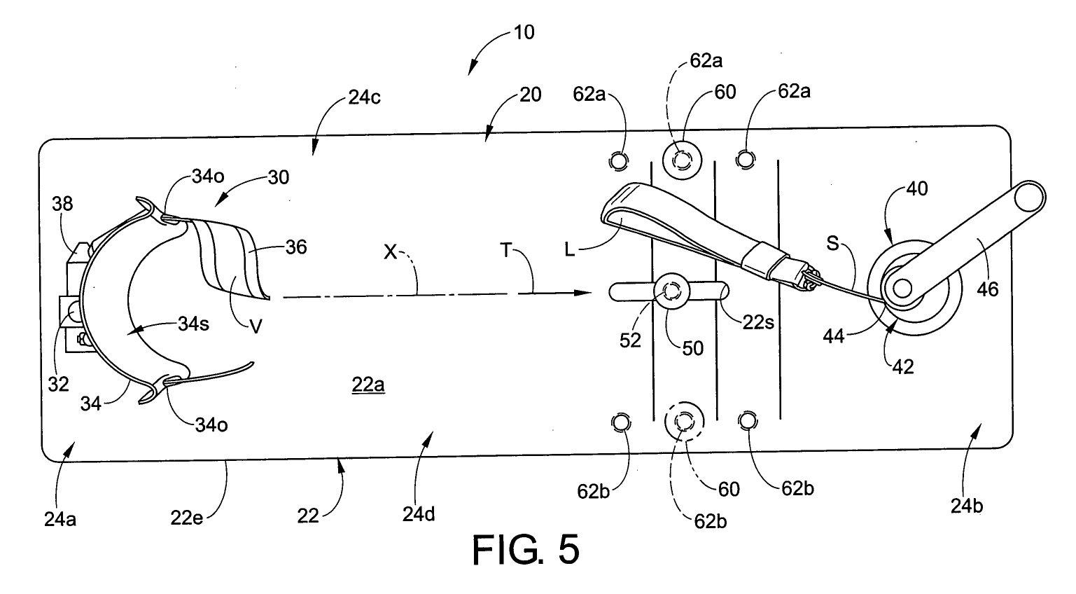

More particularly, a patient's upper arm UA is fixedly secured to the arm anchorage device 30, and at least part of the patient's forearm FA and/or hand H is supported on the base 20 (the arm anchorage device 30 is typically set at a height above the base 20 so that the patient's elbow E is slightly spaced above the base 20 while the patient's wrist W and hand H are supported on the base 20). It should be noted that the term “upper arm” UA as used herein is intended to encompass the elbow E and all arm portions inward/proximal to same. As such, the arm anchorage device can alternatively be configured to engage the elbow E. A traction applicator such as a cord and/or band and/or web and/or strap and/or polymeric member S (referred to herein as a “traction strap” without regard to whether a strap or cord or band or web or polymeric member other structure or combination of these is used) comprises a loop L that is placed around the patient's fractured wrist W. The traction strap S can alternatively comprise other structure/means for engaging the patient's wrist W and/or for applying traction to the distal radius fracture. In one alternative embodiment, shown in FIG. 3A, an alternative traction strap S′ comprises one or more finger cuffs C as are known in the art and that are connected to the strap S′ and that are engageable with one or a plurality of the patient's fingers F or thumb B (FIG. 5) to allow the longitudinal traction force to be applied to the distal radius fracture. The traction strap S is operatively coupled to the traction device 40, and the traction device 40 is used to tension the traction strap S so that the tensile force is transferred to the patient's forearm FA as a longitudinal traction force. As shown herein, it is preferred that the device 10 be configured as disclosed herein so that, in use, the patient's forearm FA is arranged transverse relative to the patient's upper arm UA, i.e., that the patient's elbow E is bent.

In the illustrated embodiment, the traction device 40 comprises a tensioner or other means for selectively tensioning the traction strap S. As shown, the tensioner comprises a winch 42 fixedly secured to the base 20, and the strap S is wound on a rotatable spool 44 of the winch 42. The winch 42 comprises a handle 46 by which a physician or other healthcare provider can rotate the spool 44 to wind the strap S onto the spool 44 to provide the required longitudinal traction force. The winch 42 includes a selectively activated ratchet, brake and/or other unidirectional rotation mechanism that, when activated, allows the spool 44 to rotate in a single direction only, i.e., in a direction to wind the strap S onto the spool 44. Of course, the winch 42 includes means for reversing the spool 44 and/or for allowing the spool to “freewheel” or rotate freely so that the strap S can be pulled from the spool to terminate or at least lessen the traction force and/or to facilitate initial set-up and engagement of the strap S with the patient's forearm FA using the loop L or other suitable means. The traction device 40 is selectively operable to apply a longitudinal traction force T to the patient's forearm via strap S against the resistance of the arm anchorage device 30.

The base 20, itself, comprises a rigid platform 22 comprising an upper surface 22a and an opposite lower surface 22b. The upper surface is flat or sufficiently flat to define a comfortable support surface for supporting a patient's forearm FA. As shown, the platform 22 comprises a peripheral edge 22e that defines a closed geometric shape which in the illustrated embodiment defines a rectangle including first and second opposite ends 24a,24b and first and second opposite lateral sides 24c,24d. As shown, the arm anchorage device 30 is connected adjacent the first end 24a and the traction device 40 is connected adjacent the second end 24b of the platform 22, and these devices 30,40 are generally aligned with each other along a longitudinal axis X (FIG. 5) of the platform 22 to allow the traction force T generated by the traction device 40 to also be aligned with the axis X. The platform 22 of the base 20 is preferably defined from a radiolucent material, at least in the areas upon which a patient's forearm is to be supported to allow radiological imaging of the patient's forearm FA even while the forearm is operatively engaged with the device. Of course, imaging in such a manner allows the status of the distal radius fracture and reduction of same to be monitored without requiring the patient's arm A to be removed from the device 10 and without requiring the traction force T to be interrupted. It is contemplated that the device 10 be used with a fluoroscope or other imaging apparatus that allows the traction force T of device 40 and any palmer translation force to be adjusted during the imaging operation for real-time adjustment and monitoring. Suitable materials for the platform 22 include radiolucent plastic (i.e., polymeric) materials such as high-density polyethylene (HDPE) and others known in the art. In the illustrated embodiment, the base 20 further comprises a plurality of adjustable support feet 20f, but these can be omitted without departing from the overall scope and intent of the present invention. The upper surface 22a of the platform can include indicia 22i that indicates distance, angles or other parameters to assist a physician or technician in properly locating a patient's forearm FA and/or in assessing the amount and direction of traction force to be applied.

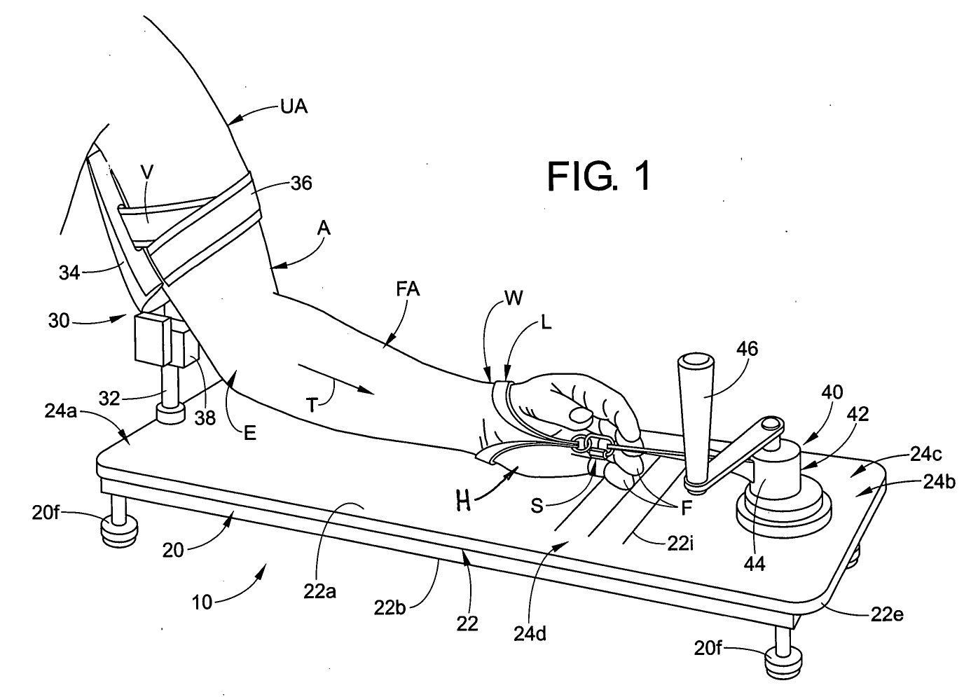

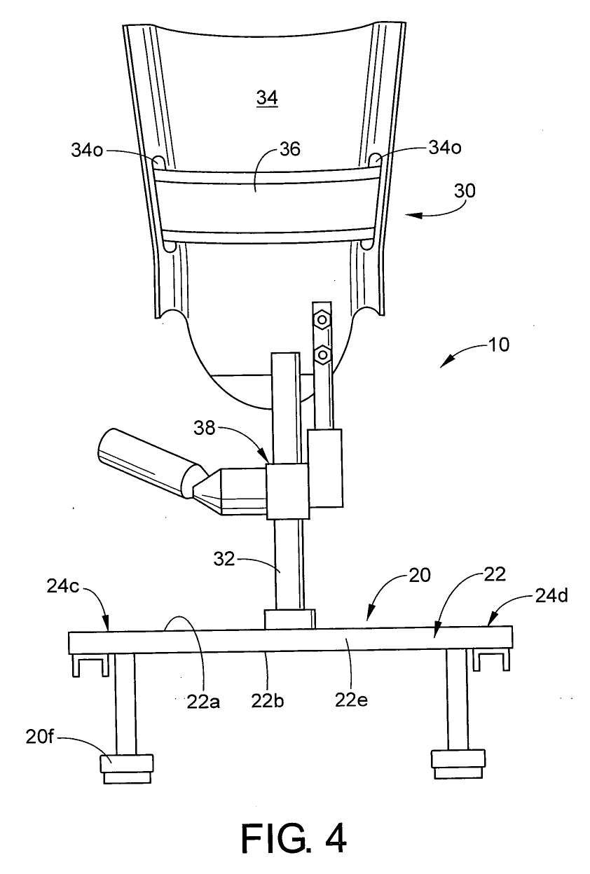

The arm anchorage device 30 comprises a post 32 that is fixedly secured to the platform 22 and that projects outwardly from the upper surface 22a. As shown the post 32 is aligned with the longitudinal axis X. The platform 22 can optionally include a plurality of different threaded or other mounting locations for fixed securement of the post 32 on either lateral side of the axis X. A cradle 34 is connected to the post 32 and is adapted to comfortably receive and support the patient's upper arm UA. As such, the cradle 34 preferably defines a U-shaped support surface 34s as best seen in FIG. 5. An upper arm securement/retention strap 36 or other device for immovably securing the patient's arm to the cradle 34 is provided. As shown the strap 36 is threaded through openings 36o (FIG. 4) defined in the cradle 34 and includes a buckle, snap hook-and-loop element and/or other fastening means for being made fast around the patient's upper arm UA. The illustrated strap 36 comprises a hook-and-loop fastening element V secured to its opposite ends and/or faces so that the strap can be wrapped around a patient's upper arm UA and fastened to itself to hold the upper arm firmly and immovably against the cradle 34 as shown in FIG. 1. As may be seen clearly in FIG. 4, the cradle 34 is connected to the post 32 by a clamp 38 that is selectively loosened to allow the height of the cradle 34 above the platform upper surface 22a to be adjusted and that is selectively tightened to hold the cradle at a desired height above the platform upper surface 22a. Clamp 38, when loosened, allows the cradle 34 to be moved pivotally toward and away from traction device 40 about a horizontal axis which allows the position of the cradle 34 to be adjusted for comfort of a particular patient. FIG. 1 also illustrates a preferred height for the cradle 34 above the platform upper surface 22a, where the patient's elbow E is spaced above the platform upper surface to facilitate splinting/casting operations while the patient's arm A is engaged with the device 10 and under traction.

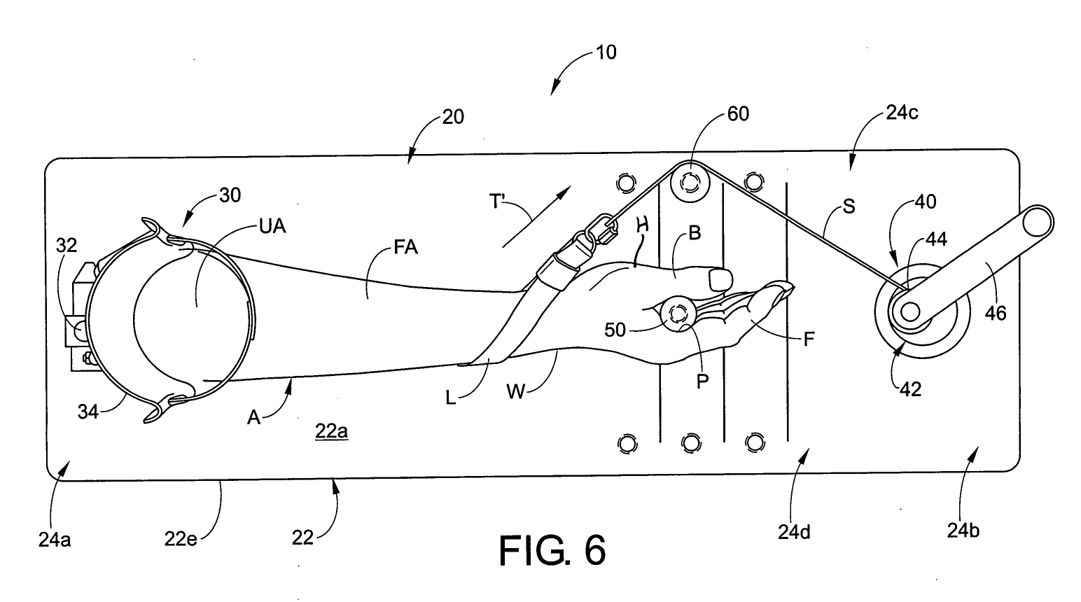

With particular reference now to FIGS. 5 and 6, the device 10 preferably comprises a forearm stabilization post 50 projecting outwardly from upper surface 22a of the platform 22 and located in alignment with the axis X and adapted to be received between a patient's palm P and thumb B (as shown in FIG. 6). The forearm stabilization post 50 helps ensure proper orientation and stabilization of the patient's hand H and forearm FA during use of the device 10. The position of the post 50 is preferably adjustable along the axis X, i.e., toward and away from the arm anchorage device 30, to accommodate different length patient forearms. As shown, the platform 22 defines an elongated slot 22s and a nut 52 is slidably connected to the platform in the slot 22s. The post 50 is threadably engaged with the nut 52 and, when tightened into the nut, the post 50 is frictionally engaged with the platform upper surface 22a and unable to move. When the post 50 is loosened in the nut 52, the post 50 and nut 52 are slidable in the slot 22s to the desired location and the post 50 is then re-tightened in the nut 52 to fix the post in the selected location. As shown in FIG. 1, use of the post 50 is optional and, as such, it can be completely unthreaded from the nut 52 and removed from the device 10.

With reference again to FIG. 5, it is often desirable to apply traction to the patient's forearm in an oblique direction relative to the longitudinal axis X. As such, the device 10 comprises a palmer translation post 60 that is optionally connected to the platform 22 in a position where the post 60 projects outwardly from platform upper surface 22a and laterally offset on one or the other side of the axis X. The post 60, when installed, is used to deflect the traction strap S as shown in FIG. 6 so that the traction force applied by the strap S when tensioned by the traction device 40 is oblique relative to the axis X as indicated by the arrow T′. This oblique traction force T′ facilitates/provides for palmer translation of the distal radius fracture. When the palmer translation post 60 is installed and used and described, the forearm stabilization post 50 is preferably used to maintain the proper position of the patient's hand H. For a wide variety of reasons, including the characteristics of the fracture, whether the fracture occurred in the patient's left or right arm, arm size, etc., it is necessary and desirable that the position of the palmer translation post 60 be adjustable. As shown, the platform 22 comprises a plurality of post mounting locations 62a,62b on respective opposite sides of the axis X into which the post 60 can be threadably or otherwise secured for use as described and as shown in FIG. 6. An example alternative location for post 60, adjacent lateral side 24d of platform 22, is shown in FIG. 5 using phantom lines.

In use, as shown in FIG. 1 and/or FIG. 6, a patient's upper arm UA is secured to the cradle 34 and the position of the cradle 34 is adjusted using clamp 38. The patient's forearm FA is supported on the upper surface 22a of the platform, preferably with the elbow E elevated above the surface 22a. If desired, the forearm stabilization post 50 and/or the palmer translation post 60 is/are installed and adjusted, and the traction strap S is operatively engaged with the patient's forearm FA by placement of the loop L around the patient's wrist. The traction force T or T′ is applied to the patient forearm FA to reduce the fracture by tensioning the strap S using the traction device 40. A splint and/or cast is then installed on the patient's forearm FA, directly over the loop L and while the traction force T or T′ is present. The loop L is then cut or otherwise separated from the strap S and stays with the patient as part of the splint/cast. When the loop L is cut or otherwise separated from the strap S, any free end thereof is preferably taped or otherwise bound to the patient's forearm as part of the splint/cast and this can help to stabilize the fracture during the healing process.

The invention has been disclosed with reference to the preferred embodiment(s). Alterations and modification will occur to those of ordinary skill in the art, and it is intended that the invention as defined by the claims be construed as broadly as legally possible to encompass all such modifications and alterations.

1. A stabilization and traction apparatus comprising:
a base;
an arm anchorage device connected to the base, said arm anchorage device adapted to engage an associated patient upper arm;
a traction device connected to the base, said traction device comprising a traction strap connected thereto and comprising a tensioner for selectively tensioning the strap.

2. The apparatus as set forth in claim 1, wherein said base comprises a platform including an upper surface for supporting at least one of: (i) an associated patient hand; (ii) an associated patient wrist; (iii) at least part of an associated patient forearm.

3. The apparatus as set forth in claim 2, wherein said platform comprises first and second opposite ends, said arm anchorage device located adjacent said first end of said platform and said traction device located adjacent said second end of said platform.

4. The apparatus as set forth in claim 3, wherein said arm anchorage device and said traction device are aligned with each other on a longitudinal axis of the platform.

5. The apparatus as set forth in claim 4, wherein said platform comprises indicia to assist locating the associated patient forearm on the platform.

6. The apparatus as set forth in claim 4, wherein the platform comprises a radiolucent material.

7. The apparatus as set forth in claim 4, further comprising a forearm stabilization post projecting outwardly from the upper surface of the platform, said forearm stabilization post adapted to be received between an associated patient thumb and associated patient palm.

8. The apparatus as set forth in claim 7, wherein said forearm stabilization post is located in alignment with the longitudinal axis of the platform.

9. The apparatus as set forth in claim 7, wherein a position of said post is selectively adjustable toward and away from said arm anchorage device.

10. The apparatus as set forth in claim 7, further comprising a palmer translation post connected to and projecting outwardly from the platform, wherein said palmer translation post is adapted to engage and deflect said traction strap between said arm anchorage device and said traction device.

11. The apparatus as set forth in claim 3, further comprising a palmer translation post connected to and projecting outwardly from the platform, wherein said palmer translation post is adapted to engage and deflect said traction strap between said arm anchorage device and said traction device.

12. The apparatus as set forth in claim 1, wherein said traction strap comprises at least one of a strap, cord, band, web, polymeric member.

13. The apparatus as set forth in claim 12, wherein said tensioner comprises a rotatable spool on which said traction strap is wound.

14. The apparatus as set forth in claim 13, wherein said tensioner further comprises a unidirectional rotation mechanism that allows rotation of said spool in a single direction only.

15. The apparatus as set forth in claim 1, wherein said traction strap comprises one of: (i) a loop for encircling the associated patient wrist; or, (ii) a plurality of finger cuffs for engaging associated patient fingers.

16. The apparatus as set forth in claim 1, wherein said arm anchorage device comprises a post that projects outwardly from the base and a cradle connected to the post, said cradle adapted to engage the associated patient upper arm.

17. The apparatus as set forth in claim 16, further comprising an upper arm securement strap for releasably securing the associated patient upper arm to the cradle.

16. The apparatus as set forth in claim 16, wherein a height of said cradle above said base is adjustable.

17. The apparatus as set forth in claim 1, wherein said base and said arm anchorage device are located relative to each other so that said base is adapted to support an associated patient forearm in a transverse orientation relative to the associated patient upper arm.

18. A method for non-operative treatment of an upper extremity fracture, said method comprising:
supporting on a base at least one of: (i) a patient hand; (ii) a patient wrist; (iii) at least part of a patient forearm;
engaging a patient upper with arm anchorage device that is connected to the base;
connecting a traction strap of a traction device to at least one of: (i) the patient hand; (ii) the patient wrist;
operating the traction device to tension the traction strap, wherein the arm anchorage device restrains the patient upper arm against movement in response to the tension in the traction strap.

19. The method as set forth in claim 18, further comprising:
applying a splint to the patient forearm while the tranction strap is tensioned and while the patient upper arm is engaged with the arm anchorage device.

20. The method as set forth in claim 18, wherein the traction device and arm anchorage device are spaced from each other on a longitudinal axis, and wherein said traction strap is routed so as to apply traction in a direction that is oblique relative to said longitudinal axis.
